(19) United States
(12) Patent Application Publication
(10) Pub. No.: US 2022/0322777 A1
(43) Pub. Date: Oct. 13, 2022

(54) HELMET SUPPORT EXOSKELETON (HSE) AND HARNESS THEREFOR

(71) Applicant: Brash Product Development Inc., Ottawa (CA)

(72) Inventors: Richard Beranek, Gatineau (CA); Aliasgar Morbi, Nepean (CA); Rohan Thakar, Ottawa (CA); Dominic Peters, Ottawa (CA); Markus Melcher, Ottawa (CA); Sava Nozin, Ottawa (CA); Robert Shudra, Ottawa (CA)

(73) Assignee: Brash Product Development Inc., Ottawa, ON (CA)

(21) Appl. No.: 17/626,870

(22) PCT Filed: Jul. 29, 2020
(86) PCT No.: PCT/CA2020/051043

§ 371 Date: Jan. 13, 2022

Related U.S. Application Data
(60) Provisional application No. 62/881,697, filed on Aug. 1, 2019.

(51) Int. Cl. A42B 3/04; A45F 3/14

(52) U.S. Cl. CPC A42B 3/0473; A45F 3/14

(57) ABSTRACT

The present invention relates to a helmet support exoskeleton and harness therefor that allow offloading the weight of head mounted equipment such as helmets and other equipment related to helmets in order to prevent and reduce neck fatigue, strain, and injuries without causing discomfort or impeding mobility of an operator. Through the use of a helmet- and harness-mounted linkage assembly, the weight is translated from an operator's head to his or her torso. This further allows the operator to wear additional and heavier equipment without causing discomforts and reduces the risk of neck injuries.

### CROSS-REFERENCE TO RELATED APPLICATIONS

This application claims priority to U.S. Provisional Application No. 62/881,697, filed on Aug. 1, 2019, the content of which is incorporated herein by reference in its entirety.

### BACKGROUND OF THE INVENTION

### Field of the Invention

The present invention relates generally to head mounted devices and body worn devices, more particularly to a helmet support exoskeleton (or linkage) and harness therefor that allow offloading the weight of head mounted equipment such as helmets and other equipment related to helmets in order to prevent and reduce neck fatigue, strain, and injuries without causing discomfort or impeding mobility of an operator, and/or to allow the operator to wear additional and heavier equipment on his/her helmet without causing discomforts and reduces the risk of neck injuries.

### Description of Related Art

Headgear is used in many fields. The most common type of headgear, a helmet is used in construction, sports, and military applications for various safety reasons. In many professions, helmets are used in conjunction with other headgear such as night vision googles (NVGs), communications equipment, and helmet mounted cameras. The additional head borne mass increases muscle exertion and neck strain and may lead to the development of chronic neck pain or injury if extension is consistent over time. This problem is compounded by devices such as NVGs where most of the weight is in front of the head center of mass, creating an unbalanced moment that the user must constantly balance. Headgear is essential in these professions; therefore, a solution is needed to prevent neck injuries.

One current solution to this problem is mounting counterbalancing weights on the back of the helmet and exercise routines to strengthen the neck muscles; however, counterweights only solve the moment issue but increase the total head borne mass and increase the head borne inertia.

Various attempts were previously made to reduce chronic neck pain or injuries caused by the head borne mass increases and/or head borne inertia. For example, Canadian patent application No. 2960415 (Fischer et al.) attempted to solve, at least, the inertia and moment issue by using a helmet-back mounted pulley system. U.S. Pat. No. 7,765,623 (Ashline) also attempted to solve, at least, the inertia and moment issue by providing a head restraint device that has a rigid spacer positioned along the back of the driver, and tethering the helmet to the vehicle's seatbelt through an anchor strap; however, Fischer et al. and Ashline are both silent about any solution to address issues related to the head-borne mass.

US Patent Application Publication No. 2017/0049177 (Margetis et al.) proposed a system and method for head and spine immobilization and protection; however, the system proposed by Margetis et al. is restrictive, and, once activated, does not allow various movement of the head in relation to the spine, thus the solution proposed by Margetis et al. would not be suitable for construction, sports or military applications.

Accordingly, there is a long-felt needs for a solution that would address at least one of the existing short-comings and problems as identified above.

### SUMMARY OF THE INVENTION

An object of the present invention is to provide means for counterbalancing any headgear induced movement, offloading head-borne mass to the torso.

Another object of the present invention is to provide means for allowing an operator to wear additional and heavier equipment on his/her helmet without causing discomforts and reduces the risk of neck injuries.

According to one aspect of the present invention, it provides devices and methods for offsetting weight from a user's helmet to the torso are disclosed herein.

According to another aspect of the present invention, it provides a device that comprises: a linkage assembly, wherein the linkage assembly comprises a helmet mount, wherein the helmet mount is associated with a wearer's helmet and a vertical arm, wherein the vertical arm is engaged with a horizontal arm, a horizontal arm associated with (iii) a lower arm, wherein the lower arm is engaged with (c) a harness designed to be secured to the user's torso.

According to yet another aspect of the present invention, it provides a helmet support exoskeleton, comprising a first linkage having first and second distal ends; a second linkage; a third linkage configured to be mounted on a harness; a first revolute joint disposed at the first distal end of the first linkage for rotatably coupling the first linkage with a helmet, and an axis of the first revolute joint is aligned with an axis of neck twist of an user; a second revolute joint for rotatably coupling the second distal end of the first linkage with the second linkage, and an axis of the second revolute joint is aligned with an axis of neck lateral bend of the user; and at least one third revolute joint for rotatably coupling the second linkage with the third linkage, and an axis of the third revolute joint is aligned with an axis of neck flexion of the user.

The third revolute joint may further comprise a torsion spring for counterbalancing a moment communicated through the first linkage to the second linkage from the helmet.

The first linkage may further comprise a first telescopic member, retractably extends from the first distal end of the first linkage; a first fastener for releasably or non-releasably locking the first telescopic member to the first linkage; a second telescopic member, retractably extends from the second distal end of the first linkage; and a second fastener for releasably or non-releasably locking the second telescopic member to the first linkage.

The helmet support exoskeleton may further comprise a mounting plate configured to be attached to the helmet and receives the first revolute joint.

The mounting plate may comprise a sliding rail for slidably receiving the first revolute joint and a fastener for releasably or non-releasably locking the first revolute joint to the sliding rail.

The third linkage may yet further comprise at least one third telescopic member coupled with the at least one third revolute joint, retractably extends from the third linkage; and a third fastener for releasably or non-releasably locking the third telescopic member to the third linkage.

The third linkage may define a slot for releasably receiving a mounting plate disposed on the harness.

The third linkage may further define a female receiver and the mounting plate on the harness further defines a corresponding female receiver for releasably receiving a pin.

The first, second and third linkages may be made of a light-weight composite.

According to further aspect of the present invention, it provides a harness for a helmet support exoskeleton, comprising a structural member interface with back of a neck and shoulder of a user and extends along the lower back of the user for receiving the helmet support exoskeleton at behind a lower portion of a cervical spine, a upper portion of a thoracic spine, or therebetween of the user; shoulder straps; abdominal straps; and waist straps; wherein the structural member defines first slots for receiving shoulder straps, second slots for receiving abdominal straps, and third slots for receiving waist straps; and wherein the shoulder straps are configured to wrap around shoulders of the user be coupled with the abdominal straps; and, the waist straps are configured to wrap around waist of the user and to be couple with each other.

The structural member may interface with a lower portion of a cervical spine and wraps around a portion of the neck and shoulder area of the user.

The lower portion of the cervical spine may be from about C5 (or fifth cervical vertebra) to C7 (or seventh cervical vertebra) of the cervical spine.

A lower portion of the structural member may terminate at a top portion of a lumbar spine of the user.

The top portion of the lumbar spine may be from L1 (or first lumbar vertebrae) to about L3 (or third lumbar vertebrae).

The harness may further comprise chest straps that is configured to wrapped over an upper chest or collarbone of the user for coupling the shoulder straps.

The harness may further comprise a mounting plate base; a mounting plate configured to be coupled with the mounting plate base for receiving the helmet support exoskeleton.

The mounting plate base may define an array of index holes for adjusting the mounting location of the mounting plate and receiving one or more fasteners for coupling the mounting plate with the mounting plate base.

The structural member may comprise a first member that interface with the lower portion of the cervical spine and wraps around the portion of the neck and shoulder area of the user and extends to the upper back in between the shoulder blades of the user; and, a second member that is connected to a lower portion of the first member and extends to the top portion of a lumbar spine of the user.

### BRIEF DESCRIPTION OF THE DRAWINGS

The present invention will be apparent from the description herein and the accompanying drawings, in which like reference characters refer to the same parts throughout the different views. The drawings are not to scale.

FIG. 1 is a rear-dimetric view showing a helmet support exoskeleton (or linkage) and harness therefor in accordance with a preferred embodiment of the present invention;

FIG. 2 is a front-dimetric view thereof;

FIG. 3 is a rear-isometric isolated view of the linkage;

FIG. 4 is a front-dimetric exploded view showing adjustability and helmet interface of linkage;

FIG. 5 is a rear-dimetric exploded view showing adjustability, quick release and linkage interface with harness;

FIG. 6 is a rear-dimetric isolated view of the harness;

FIG. 7 is a front dimetric exploded view showing adjustability of the harness; and

FIG. 8 is a rear-dimetric schematic view of the linkage mechanism.

### LIST OF REFERENCE NUMERALS

LINKAGE ASSEMBLY 1
Helmet Mount 11
   Helmet Mounting Plate 111
   Prismatic Joint A 112
   Adjustment Locking Screw 113
   Sliding Rail 114
Linkage A 12
   Body Member 121
   Prismatic Joint B 122
   Prismatic Joint C 123
   Adjustment Locking Screws 124T, 124B
   Revolute Joint A 125
   Axis A 126
   Sliding Rails 127T, 127B
   Telescopic Members 128T, 128B
Linkage B 13
   Right Portion of Linkage B 13R
   Left Portion of Linkage B 13L
   Body Member 131
   Revolute Joint B 132
   Axis B 133
   Axis AB Point of Intersection 134
Linkage Base 14
   Body Member 141
   Prismatic Joints D 142R, 142L
   Adjustment Locking Screws 143R, 143L
   Revolute Joint C 144R, 144L
   Axis C 145
   Torsion Springs 146R, 146L
   Quick Release Pin 147
   Pull Tag 148
   Sliding Rails 149R, 149L
   Telescopic Members 150R, 150L
   Female Receiver 151
   Slot 152
HARNESS 2
Structural Back 21
   Mounting Plate Base 211
   Mounting Plate 212
   Mounting Plate Locking Screws 213T, 213B
   Neck/Shoulder Component 214
   Lower Back Component 215
   Locking Screws 216T, 216B
   Array of Index Holes 217
   Corresponding Female Receiver 218
   Slots 219TR, 219TL, 219MR, 219ML, 219BR, 219BL,
   Sliding Mechanism 2101
   Array of Index Holes 2102
Strap Assembly 22
   Shoulder Straps 221R, 221L
   Waist Straps 222R, 222L
   Abdominal Straps 223R, 223L
   Chest Straps 224R, 224L
   Strap Connectors 225R, 225L
   Strap Fasteners 226T, 226B
HELMET 3
USER/OPERATOR 4

### DETAILED DESCRIPTION OF THE INVENTION

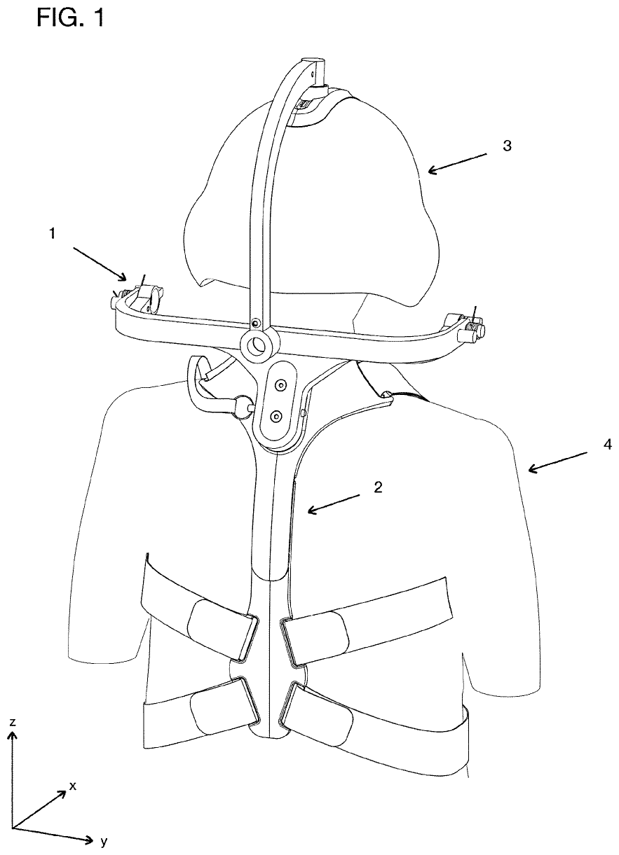

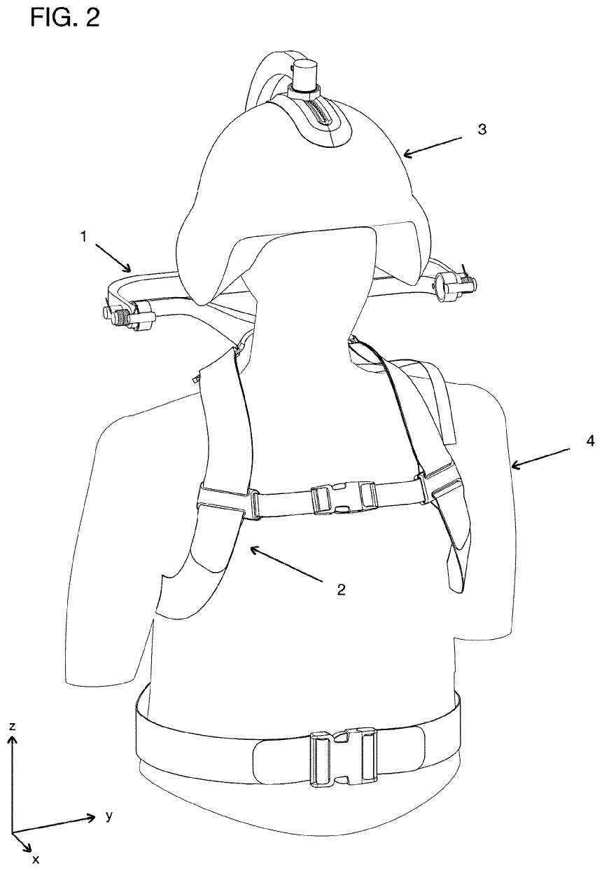

FIG. 1 and FIG. 2 show a rear perspective view and front perspective view, respectively, of a helmet support exoskeleton (or linkage assembly) and harness therefor in accordance with a preferred embodiment of the present invention, including, but not limited to, a helmet 3, linkage assembly 1 and harness 2 that is worn by a user 4.

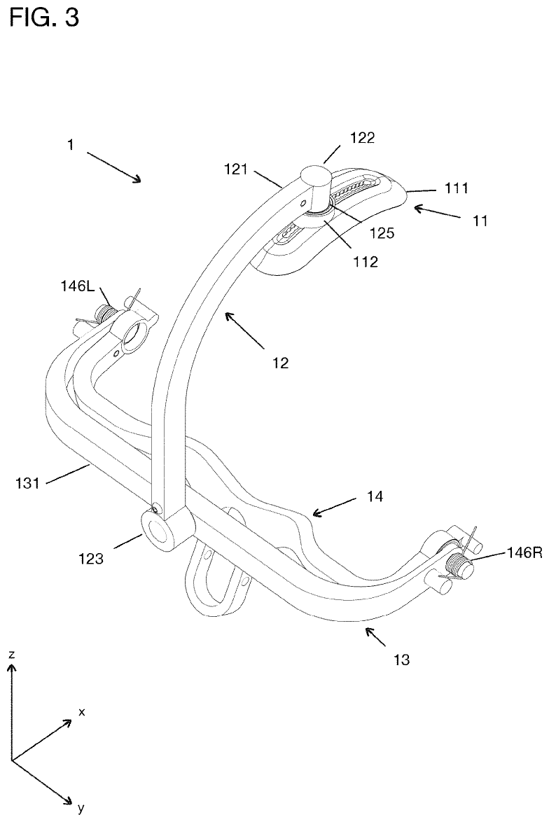

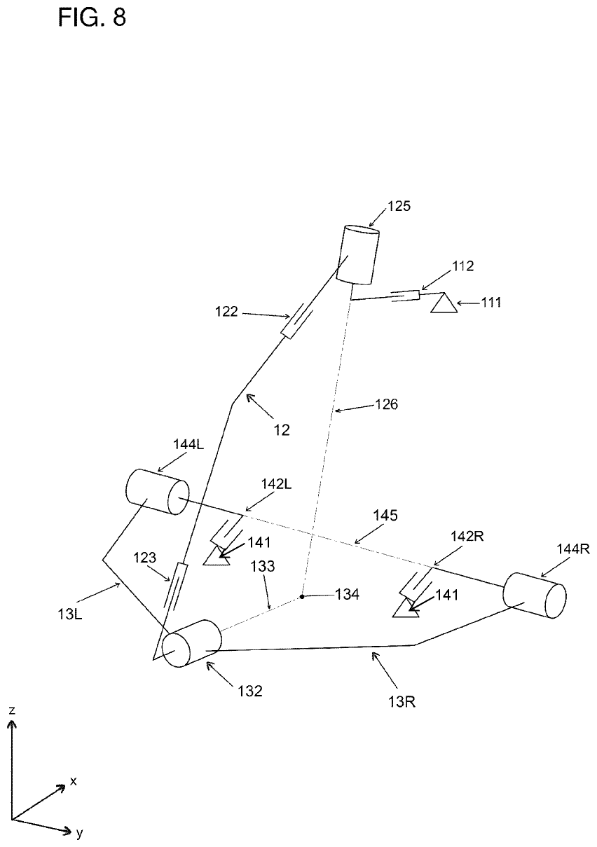

As shown in FIG. 3, the linkage 1 is composed of the helmet mount 11, linkage A 12, linkage B 13, and the linkage base 14. FIG. 8 details the linkage kinematic arrangement which is a prismatic-revolute-revolute-prismatic-prismatic-revolute-prismatic (PRRPPRP) serial chain.

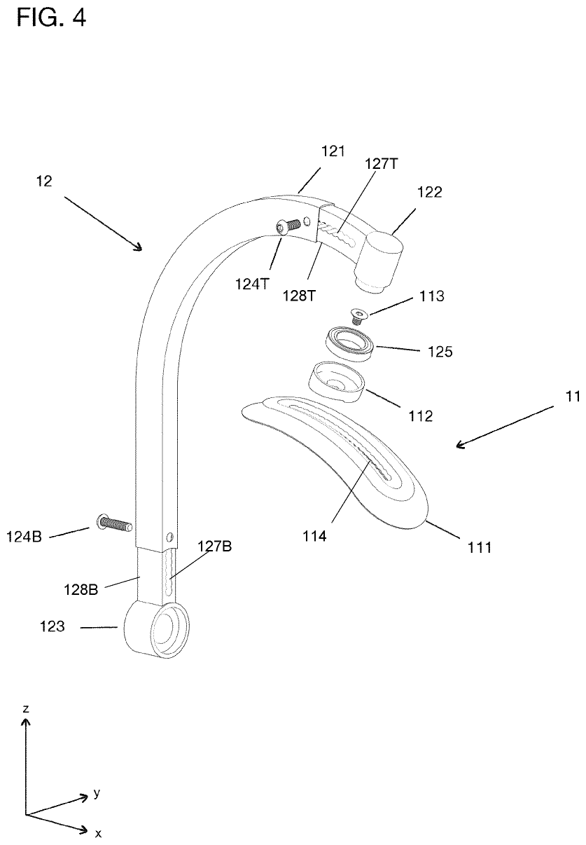

Each revolute joint is aligned with a corresponding axial movement of the cervical spine. Axis A 126 of the revolute joint A 125 is aligned with an axis of neck twist (Z-axis) of the user 4; axis B 133 of the revolute joint B 132 is aligned with an axis of neck lateral bend (X-axis) of the user 4; and, an axis of each of revolute joints C 144R, 144L is aligned with an axis of neck flexion/extension (Y-axis) of the user 4. The revolute joints shown in FIG. 4. are preferably ball bearings, but they can also be designed as bushings or compliant joints or other suitable means.

Each prismatic joint function as a point of adjustability and is locked in a certain position during use. One embodiment of the prismatic joints is depicted in FIG. 4 and consist of sliding rails 114, 127T, 127B, 149R, 149L on helmet mounting plate 111, extending/telescopic member 128T, 128B, 150R, 150L, respectively, with indexed holes to releasably or non-releasably fasten/lock the position using fasteners, such as, for example, screws 113, 124T, 124B, 143R, 143L. The prismatic joints 112, 122, 123, 142R, 142L may also be designed as telescoping tubes, scissor mechanism, lead screw or multiple sizes of the same part. In accordance with the preferred embodiment of the present invention, fasteners or locks to releasably or non-releasably lock the position of the prismatic joints along the sliding rails 114, 127T, 127B, 149R, 149L are set screws 113, 124T, 124B, 143R, 143L. The releasable or non-releasable locks may also be designed as a clamp, ratchet, or pin mechanism.

Referring to FIG. 8, the linkage A is configured such that axis A 126 of rotation of the revolute joint A 125 and axis B 133 of rotation of the revolute joint B 132 intersect at an arbitrary point (or point intersection) 134. The location of this point varies depending on the positions of the prismatic joints 122, 123 due to different physical size characteristics of a given user. Axis C 145 of rotation of the revolute joints C 144 may not always intersect with this point 134 depending on the adjustment position. The linkage base 14 and linkage B 13 may be symmetric as depicted in FIG. 8 or asymmetrical where they only consist of either the left (i.e. linkage base 14L, linkage B 13L alone) or right side (i.e. linkage base 14R, linkage B 13R alone).

Helmet mount 11 consists of a mounting plate 111. The mounting plate 111 may be secured to the helmet 3 with adhesive or fasteners, and has the sliding rail 114 for receiving the prismatic joint A 112/revolute joint A 125. Preferably, the mounting plate 111 would be secured to the helmet 3 and be oriented in such a way that the sliding rail 114 intersects with an axis of neck twist (Z-axis) of the user 4, and extends to the front and to the back of the helmet 3, and preferably, extends along on the planes defined by X-axis and Z-axis. The prismatic joint A 112 may be fastened at an arbitrary point along the sliding rail 114 for a position adjustment. Optionally, the mounting plate 111 may be integrally formed with or embedded within the helmet 3.

Linkage A 12 is composed of a body member 121 and the prismatic joint B 122 disposed at a first distal end thereof, which includes the telescopic member 128T that retractably extends from the body member 121; and the prismatic joint C 123 disposed at a second distal end thereof, which includes the telescopic member 128B that retractably extends from the body member 121. The revolute joint A 125 is disposed at the distal end of the prismatic joint B 122 for rotatably coupling the linkage A with the helmet mount 111, while the revolute joint B 132 is disposed at the distal end of the prismatic joint C 123 for rotatably coupling the linkage A 12 with the linkage B 13. The body member 121 is preferably a lightweight and rigid material such as carbon fiber composite.

Linkage B 13 is of similar composition to linkage A 12. A body member 131 of the linkage B 13 extends laterally from the revolute joint B 132 where the linkage A 12 is rotatably coupled therewith. The body member 131 uses the same materials and construction as the body member 121 of the linkage A 12.

Linkage base 14 comprises the body member 141. Prismatic joints D 142R, 142L are disposed at the distal ends of the linkage base 14, including extendible/telescopic members 150R, 150L that retractably extend from the distal ends of the body member 141 of the linkage base 14. Revolute joints C 144R, 144L are disposed at its distal ends of the telescopic members 150R, 150L, respectively, and rotatably couple the distal ends of the linkage base 14 with the corresponding distal ends of the body member 131 of the linkage B 13.

As the moment of the helmet 3 is carried/propagated through the linkage A 12 to the linkage B 13, torsion springs 146R, 146L are provided between the linkage B 13 and the linkage base 14 and function to counterbalance the moment created by any helmet mounted object such as night vision goggles or a helmet camera. The preferred embodiment of the torsion springs 146R, 146L is shown in FIG. 3. A person of ordinary skilled in the pertinent art would understand that their function of the torsion springs 146R, 146L can also be achieved by using pneumatic springs, constant force wound springs, electric motors or any other mechanism that can generate a torsional force.

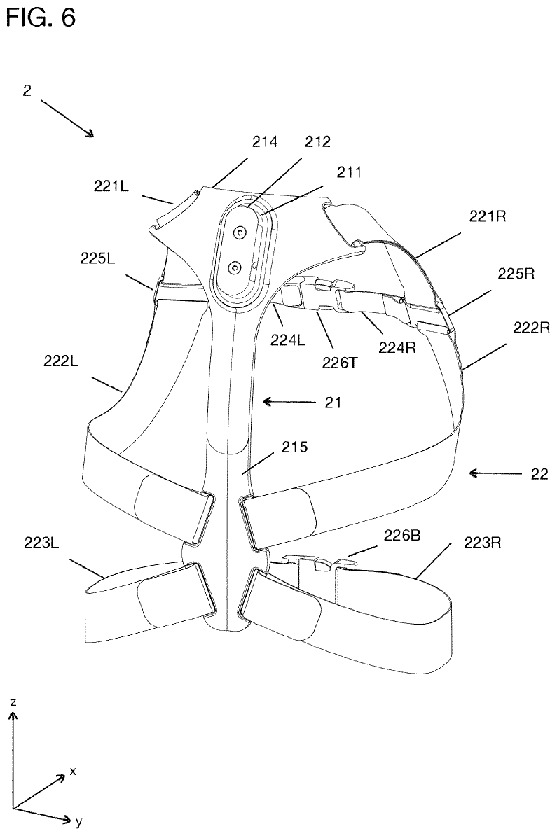

With reference to FIG. 6 the harness 2 comprises a structural back 21 and a strap assembly 22.

The structural back 21 has a narrow and thin profile to minimize interference with the shoulder blades and may be assembled from a rigid material such as metal or plastic, preferably a thermoplastic material, that further preferably is durable, lightweight and flexible which allows to adapt to various body movements. It may also consist of a combination of materials that vary in durometer. the structural back 21 may be padded along all or a portion of its length. As shown in FIG. 1 and FIG. 2 the structural back 21 is centered on the Y-axis of the users 4 back and extends along the lengthwise direction of the spine of the user 4. The structural back 21 may be a single component or include multiple components.

With reference to FIG. 6, according to the preferred embodiment of the present invention, the structural back 21 preferably comprises a neck/shoulder component/member 214 and a lower back component/member 215. The neck/shoulder component 214 interface with back of a neck and shoulder of the user 4. According to the preferred embodiment of the present invention, an upper end of the neck/shoulder component 214 starts at/interfaces with the lower portion (anywhere from about C5 (or fifth cervical vertebra) to C7 (or seventh cervical vertebra)) of the cervical spine of the user 4 where neck/shoulder component 214 wraps around a portion of the neck and shoulder area of the user 4. The width of the lower section of the neck/shoulder component 214 narrows down in order to minimize any interference with any shoulder movements. The lower section of the neck/shoulder component 214 ends on the upper back in between the shoulder blades of the user 4 where it attaches to the lower back component 215. Preferably, the bottom end of a lower back component 215 terminates at the top portion (from L1 (or first lumbar vertebrae) to about L3 (third lumbar vertebrae), preferably, at L1 or L2 (or second lumbar vertebrae) of the lumbar spine.

The structural back 21 includes several slots. For example, slots 219TR and 219TL for receiving/coupling shoulder straps 221R and 221L, respectively; slots 219MR and 219ML for receiving/coupling waist straps 222R and 222L, respectively; and, slots 219BR and 219BL for receiving/coupling abdominal straps 223R and 223L, respectively.

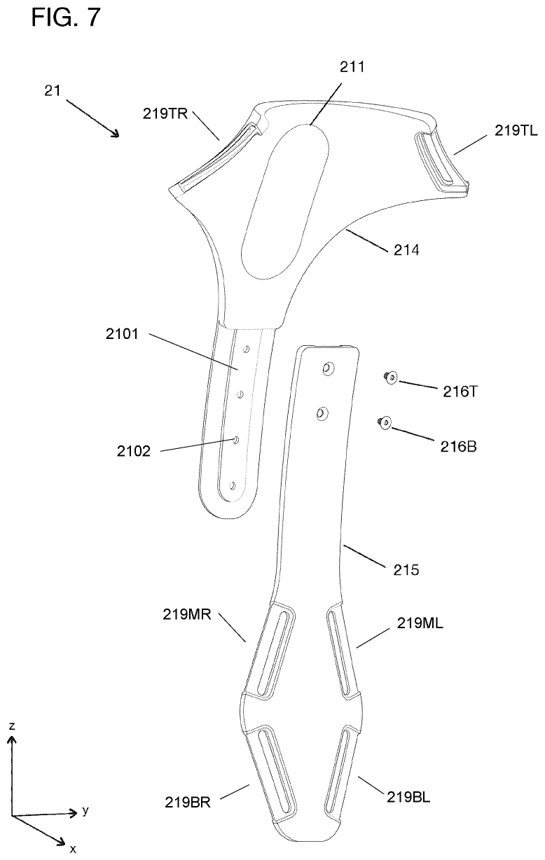

As particularly shown in FIG. 7, the structural back 21 is length-adjustable in Z-direction or along the length of the back to best fit the user's back. This may be accomplished through a sliding mechanism 2101 with an array of index holes 2102 which can be fixated through releasable/non-releasable locking screws 216T, 216B.

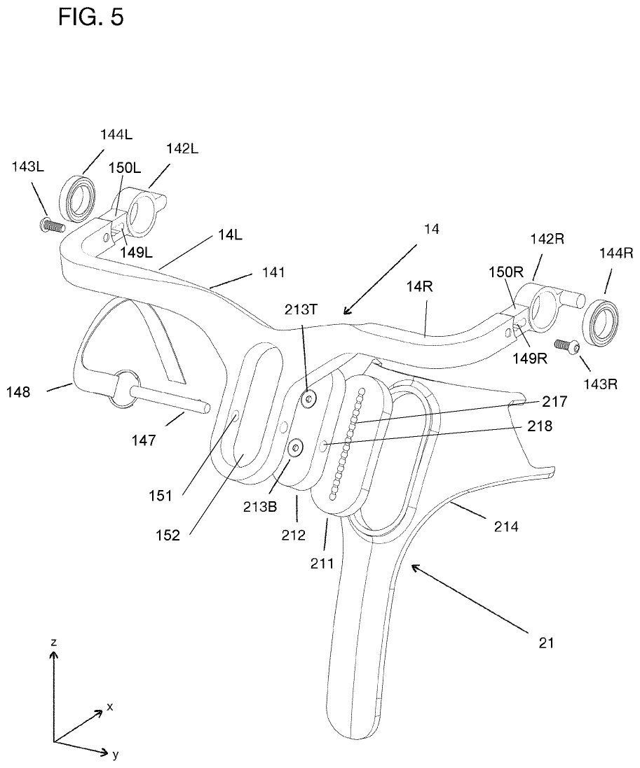

As particularly shown in FIG. 5, a mounting plate 212 is center mounted on a mounting plate base 211. Both components are preferably rigid/non-flexible and may be constructed from a lightweight and durable metal or plastic. The mounting plate base 211 may offer an array of index holes 217 along the lengthwise direction of the spine of the user 4 for allowing Z-axis adjustability in relation to a mounting plate 212. The mounting plate base 211 and mounting plate 212 may be arranged such that the mounting plate 212 would be located about behind the lower portion (from about C5 to C7) of the cervical spine, upper portion (from T1 (or first thoracic vertebrae) to about T5 (or fifth thoracic vertebrae)) of the thoracic spine, or therebetween (from about C5 to about T5) of the user 4. The mounting plate base 211 may be permanently attached to the neck/shoulder component 214, fastened by mounting plate locking screws 213T, 213B. The linkage base 14 defines a slot 152 for receiving the mounting plate 212 such that the mounting plate and the linkage base 14 interlocks with each other. The mounting plate 212 and linkage base 14 may incorporate a quick release feature, which, as shown in FIG. 5, may contain a female receiver 151 on the linkage base 14 and a corresponding female receiver 218 on the mounting plate 212 for releasably receiving a quick release pin 147. A quick release pin 147 is attached to a pull tag 148 that is accessible to the user 4 during operation. A quick release may be implemented in various other suitable way that allows a user 4 to quickly unlock the linkage assembly 1 from the harness 2.

The strap assembly 22 comprises shoulder straps 221R, 221L, waist straps 222R, 222L, chest straps 224R, 224L and abdominal straps 223R, 223L which may be made from any suitable fabric, preferably some portions or their entire length are elasticated. Examples of suitable strap materials include, but are not limited to, synthetic textiles.

According to a preferred embodiment of the present invention, any of the shoulder straps 221R, 221L, waist straps 222R, 222L, chest straps 224R, 224L and abdominal straps 223R, 223L, some portions thereof or their entire lengths, may optionally be provided with additional padding to increase comfort during operation 4. The strap assembly 22 may be removably or permanently connected to a structural back 21. In order to configure the strap assembly 22 to the size of the user 4, the straps including shoulder straps 221R, 221L, waist straps 222R, 222L, chest straps 224R, 224L and abdominal straps 223R, 223L may include length adjusters such as a VELCRO® fastener (not shown) or any other device or means providing a length-adjustment capability. Such a length-adjusting capability may be provided at the strap fasteners 226T, 226B and/or strap connectors 225R, 225L, which structures are well understood and known among ordinary skilled in the art. Strap connectors 225R, 225L link the waist straps 222R, 222L, shoulder straps 221R, 221L and chest straps 224R, 224L, respectively. Chest straps 224R, 224L and abdominal straps 223R, 223L use strap fasteners 226T, 226B, respectively, which may be any suitable fastener capable of allowing adjustment of the straps, such as buckles, side release or cam buckles.

With reference to FIG. 6, according to a preferred embodiment of the present invention, ends of shoulder straps 221R, 221L and one ends of waist straps 222R, 222L fixedly attach at the structural back 21 and extend to strap connectors 225R, 225L which are located in the upper chest/collarbone area of the user 4. Similarly, proximal ends of abdominal straps 223R, 223L engage with the structural back 21 and the distal ends terminate with strap fastener 226B. In parallel, ends of the chest straps 224R, 224L engage with the strap connectors 225R, 225L and terminate with the strap fastener 226T which preferably offers strap length adjustability. A strap assembly 22 may include a portion of VELCRO® strap fasteners (not shown).

When worn by a user 4, the waist straps 222R, 222L can be adjusted by pulling the ends near the strap connectors 225R, 225L and fasten it at any point on the waist straps 222R, 222L. The waist straps 222R, 222L can also be completely detached from the strap connectors 225R, 225L when the harness 2 is taken on or off by the user 4.

It is to be understood that a person of ordinary skilled in the art would realize and understand that there are variations or various modifications that can be applied to the presently described exemplary embodiments without departing from the spirit of the present invention. Accordingly, the specification and drawings should not be considered as restrictive, but rather be regarded as illustrative.

1. A helmet support exoskeleton, comprising:
a first linkage having first and second distal ends;
a second linkage;
a third linkage configured to be mounted on a harness;
a first revolute joint disposed at the first distal end of the first linkage for rotatably coupling the first linkage with a helmet, and an axis of the first revolute joint is aligned with an axis of neck twist of an user;
a second revolute joint for rotatably coupling the second distal end of the first linkage with the second linkage, and an axis of the second revolute joint is aligned with an axis of neck lateral bend of the user; and
at least one third revolute joint for rotatably coupling the second linkage with the third linkage, and an axis of the third revolute joint is aligned with an axis of net flexion of the user.

2. The helmet support exoskeleton as recited in claim 1, wherein the third revolute joint further comprising a torsion spring for counterbalancing a moment communicated through the first linkage to the second linkage from the helmet.

3. The helmet support exoskeleton as recited in claim 1, wherein the first linkage further comprising:
a first telescopic member, retractably extends from the first distal end of the first linkage;
a first fastener for releasably or non-releasably locking the first telescopic member to the first linkage;
a second telescopic member, retractably extends from the second distal end of the first linkage; and
a second fastener for releasably or non-releasably locking the second telescopic member to the first linkage.

4. The helmet support exoskeleton as recited in claim 1 further comprising a mounting plate configured to be attached to the helmet and receives the first revolute joint.

5. The helmet support exoskeleton as recited in claim 4, wherein the mounting plate comprising a sliding rail for slidably receiving the first revolute joint and a fastener for releasably or non-releasably locking the first revolute joint to the sliding rail.

6. The helmet support exoskeleton as recited in claim 1, wherein the third linkage further comprising:
at least one third telescopic member coupled with the at least one third revolute joint, retractably extends from the third linkage; and
a third fastener for releasably or non-releasably locking the third telescopic member to the third linkage.

7. The helmet support exoskeleton as recited in claim 1, wherein the third linkage defines a slot for releasably receiving a mounting plate disposed on the harness.

8. The helmet support exoskeleton as recited in claim 7, wherein the third linkage further defines a female receiver and the mounting plate on the harness further defines a corresponding female receiver for releasably receiving a pin.

9. The helmet support exoskeleton as recited in claim 1, wherein the first, second and third linkages are made of a light-weight composite.

10. A harness for a helmet support exoskeleton, comprising:
a structural member interface with back of a neck and shoulders of a user and extends along the lower back of the user for receiving the helmet support exoskeleton at behind a lower portion of a cervical spine, a upper portion of a thoracic spine, or therebetween of the user;
shoulder straps;
abdominal straps; and
waist straps;
wherein the structural member defines first slots for receiving shoulder straps, second slots for receiving abdominal straps, and third slots for receiving waist straps;
wherein the shoulder straps are configured to wrap around shoulders of the user be coupled with the abdominal straps; and, the waist straps are configured to wrap around waist of the user and to be couple with each other.

11. The harness as recited in claim 10, wherein the structural member interfaces with a lower portion of a cervical spine and wraps around a portion of the neck and shoulder area of the user.

12. The harness as recited in claim 11, wherein the lower portion of the cervical spine is from about C5 to C7 of the cervical spine.

13. The harness as recited in claim 10, wherein a lower portion of the structural member terminates at a top portion of a lumbar spine of the user.

14. The harness as recited in claim 13, wherein the top portion of the lumbar spine is from L1 to about L3.

15. The harness as recited in claim 10 further comprising chest straps that is configured to wrapped over an upper chest or collarbone of the user for coupling the shoulder straps.

16. The harness as recited in claim 10 further comprising a mounting plate base; a mounting plate configured to be coupled with the mounting plate base for receiving the helmet support exoskeleton.

17. The harness as recited in claim 16, wherein the mounting plate base defines an array of index holes for adjusting the mounting location of the mounting plate and receiving one or more fasteners for coupling the mounting plate with the mounting plate base.

18. The harness as recited in claim 10, wherein the structural member comprises:
a first member that interface with the lower portion of the cervical spine and wraps around the portion of the neck and shoulder area of the user and extends to the upper back in between the shoulder blades of the user; and
a second member that is connected to a lower portion of the first member and extends to the top portion of a lumbar spine of the user.
